SEQUENCE LISTING (34 sequences)

SEQ ID NO: 1 - protein, 340 aa, synthetic construct
  REGION 1..340: note = hsSB
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEASKPKNTIPTVKHGGGSIMLWGCFAAGGTGALHKIDGSMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 2 - protein, 340 aa, synthetic construct
  REGION 1..340: note = non mutated SB100X
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 3 - protein, 343 aa, synthetic construct
  REGION 1..343: note = hsSB for recombinant expression
GPMMGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEASKPKNTIPTVKHGGGSIMLWGCFAAGGTGALHKIDGSMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 4 - DNA, 27 bp, Homo sapiens
  source 1..27: mol_type = genomic DNA
tgtatatatatatatacagttgaagtc

SEQ ID NO: 5 - DNA, 27 bp, Homo sapiens
  source 1..27: mol_type = genomic DNA
gacacatacatacatacagttgaagtc

SEQ ID NO: 6 - DNA, 27 bp, Homo sapiens
  source 1..27: mol_type = genomic DNA
ctgttggatgcctctacagttgaagtc

SEQ ID NO: 7 - DNA, 27 bp, Homo sapiens
  source 1..27: mol_type = genomic DNA
gatatatacatatgtacagttgaagtc

SEQ ID NO: 8 - DNA, 27 bp, Homo sapiens
  source 1..27: mol_type = genomic DNA
gtactgagtgtatgtacagttgaagtc

SEQ ID NO: 9 - DNA, 27 bp, Homo sapiens
  source 1..27: mol_type = genomic DNA
ctttcaggaacaaatacagttgaagtc

SEQ ID NO: 10 - DNA, 28 bp, Homo sapiens
  source 1..28: mol_type = genomic DNA
tcaacttcagaaatgtacagttgaagtc

SEQ ID NO: 11 - DNA, 28 bp, Homo sapiens
  source 1..28: mol_type = genomic DNA
ggacacatacatacatacagttgaagtc

SEQ ID NO: 12 - DNA, 27 bp, Homo sapiens
  source 1..27: mol_type = genomic DNA
gtactgagtgtatgtacagttgaagtc

SEQ ID NO: 13 - DNA, 26 bp, Homo sapiens
  source 1..26: mol_type = genomic DNA
tttcaggaacaaatacagttgaagtc

SEQ ID NO: 14 - DNA, 27 bp, Homo sapiens
  source 1..27: mol_type = genomic DNA
actctcctatgatatacagttgaagtc

SEQ ID NO: 15 - DNA, 27 bp, Homo sapiens
  source 1..27: mol_type = genomic DNA
actctcctatgatatacagttgaagtc

SEQ ID NO: 16 - DNA, 27 bp, Homo sapiens
  source 1..27: mol_type = genomic DNA
aataatgctagttatacagttgaagtc

SEQ ID NO: 17 - DNA, 27 bp, Homo sapiens
  source 1..27: mol_type = genomic DNA
atggcgagttaacatacagttgaagtc

SEQ ID NO: 18 - DNA, 27 bp, Homo sapiens
  source 1..27: mol_type = genomic DNA
tattccatggcatatacagttgaagtc

SEQ ID NO: 19 - DNA, 27 bp, Homo sapiens
  source 1..27: mol_type = genomic DNA
tatagctaacaatatacagttgaagtc

SEQ ID NO: 20 - DNA, 26 bp, Homo sapiens
  source 1..26: mol_type = genomic DNA
gcaagtcctgtcatacagttgaagtc

SEQ ID NO: 21 - DNA, 27 bp, Homo sapiens
  source 1..27: mol_type = genomic DNA
ttaaatggaataattacagttgaagtc

SEQ ID NO: 22 - DNA, 27 bp, Homo sapiens
  source 1..27: mol_type = genomic DNA
gacacatacatacatacagttgaagtc

SEQ ID NO: 23 - DNA, 27 bp, Homo sapiens
  source 1..27: mol_type = genomic DNA
aaagcaatagcacatacagttgaagtc

SEQ ID NO: 24 - DNA, 27 bp, Homo sapiens
  source 1..27: mol_type = genomic DNA
ttgtataaatcatatacagttgaagtc

SEQ ID NO: 25 - DNA, 27 bp, Homo sapiens
  source 1..27: mol_type = genomic DNA
tatagctaacaatatacagttgaagtc

SEQ ID NO: 26 - DNA, 27 bp, Homo sapiens
  source 1..27: mol_type = genomic DNA
cctaatcatctacttacagttgaagtc

SEQ ID NO: 27 - DNA, 27 bp, Homo sapiens
  source 1..27: mol_type = genomic DNA
ttgtataaatcatatacagttgaagtc

SEQ ID NO: 28 - DNA, 27 bp, Homo sapiens
  source 1..27: mol_type = genomic DNA
catgtcacatgaagtacagttgaagtc

SEQ ID NO: 29 - DNA, 27 bp, Homo sapiens
  source 1..27: mol_type = genomic DNA
agtgaggtttaacatacagttgaagtc

SEQ ID NO: 30 - DNA, 27 bp, Homo sapiens
  source 1..27: mol_type = genomic DNA
ctattttggaaacatacagttgaagtc

SEQ ID NO: 31 - DNA, 54 bp, Homo sapiens
  source 1..54: mol_type = genomic DNA
catgtcacatgaagtacagttgaagtcgacttcaactgtacattaggtaaccac

SEQ ID NO: 32 - DNA, 55 bp, Homo sapiens
  source 1..55: mol_type = genomic DNA
attgtataaatcatatacagttgaagtcgacttcaactgtatctacatattcata

SEQ ID NO: 33 - DNA, 54 bp, Homo sapiens
  source 1..54: mol_type = genomic DNA
tcttttgttgcatatacagttgaagtcgacttcaactgtatgtatgcatttctg

SEQ ID NO: 34 - DNA, 54 bp, Homo sapiens
  source 1..54: mol_type = genomic DNA
ccctctcctacacatacagttgaagtcgacttcaactgtacattatactactaa

(19) United States
(12) Patent Application Publication
(10) Pub. No.: US 2024/0287480 A1
(43) Pub. Date: Aug. 29, 2024

(54) USE OF AN IMPROVED SLEEPING BEAUTY TRANSPOSASE WITH INCREASED PROTEIN SOLUBILITY TO FACILITATE AND CONTROL TRANSFECTION OF A TARGET CELL

(71) Applicant: European Molecular Biology Laboratory, Heidelberg (DE)

(72) Inventors: Orsolya BARABAS, Gaiberg (DE); Irma QUERQUES, Zurich (CH); Cecilia Ines ZULIANI, Heidelberg (DE)

(21) Appl. No.: 18/650,206

(22) Filed: Apr. 30, 2024

Related U.S. Application Data
(63) Continuation of application No. 17/817,801, filed on Aug. 5, 2022.
Application No. 17/817,801 is a continuation of application No. 16/640,976, filed on Feb. 21, 2020, now Pat. No. 11,441,132.

(30) Foreign Application Priority Data
Aug. 21, 2017 (EP) 17187128.8

(51) Int. Cl. C12N 9/12

(52) U.S. Cl. CPC C12N 9/1241

(57) ABSTRACT

The present invention relates to improved transposase polypeptides having increased solubility. The enzyme of the invention was developed based on the Sleeping Beauty (SB) transposase. The invention provides further nucleic acids, vectors and recombinant cells encoding or containing the improved transposase, as well as a transposase system. Furthermore provided are medical and non-medical uses of the transposase of the invention for gene delivery. The invention is in particular useful as a tool for gene delivery in genetically modified cell based therapeutic approaches for treating various diseases.

The present invention relates to improved transposase polypeptides having increased solubility. The enzyme of the invention was developed based on the Sleeping Beauty (SB) transposase. The invention provides further nucleic acids, vectors and recombinant cells encoding or containing the improved transposase, as well as a transposase system. Furthermore provided are medical and non-medical uses of the transposase of the invention for gene delivery. The invention is in particular useful as a tool for gene delivery in genetically modified cell based therapeutic approaches for treating various diseases.

### BACKGROUND OF THE INVENTION

DNA transposons are discrete genetic entities ubiquitously spread across the tree of life that can move within and between genomes. They are prominent evolutionary forces fostering genome remodeling, evolutionary changes, transmission of antibiotic resistance determinants, and the development of new biological functions such as adaptive immunity. Due to their natural properties, DNA transposons have been successfully utilized as artificial gene carriers and insertional mutagens in transgenesis and functional genomics.

The use of DNA transposons for genome manipulations in vertebrates was first enabled by the reconstruction of the Sleeping Beauty (SB) transposon from the genomes of salmonid fish. The applied SB transposon system consists of a transposon, made up of a gene of interest (genetic cargo) flanked by the specific SB inverted repeats (IRs), and the transposase protein expressed from a separate plasmid or locus. The transposase specifically binds to the IRs, cuts the transposon from a donor locus and integrates it in a new genomic location. SB has exceptionally high insertion efficiency in vertebrate genomes, which has allowed its development into a prime genetic tool, successfully applied in transgenesis of higher organisms, stem cell generation and cancer gene discovery.

Importantly, SB is now also applied as non-viral gene delivery vector in a number of clinical trials five of which aim to ex vivo modify T cells by incorporating a chimeric antigen receptor (CAR) against malignancy-specific antigens. In these studies, the SB transposase inserts a CAR gene-carrying transposon from a donor plasmid into the genome of patient-derived T cells, which are successively re-infused in the cancer patient. The introduced CARs provide the T cells with new specificities to distinctively target the cancer cells and trigger effector functions upon antigen encounter. Most successful CAR-T therapies target the CD19 antigen that is overexpressed in malignant B cells. This therapy has shown unprecedented response rates (70%-90%) in the treatment of acute and chronic leukemia and will likely enter mainstream care for many B cell malignancies in the next years. However, for the treatment of large number of patients, there is a pressing need to improve manufacturing feasibility and safety, which are also critical requirements of gene therapy in general.

Because SB is a simple binary synthetic system, it is cheaper, easier and faster to produce and implement than viral vectors. This provides a particular advantage especially for single-use and personalized applications. Use of the non-viral SB vector also reduces the risks of undesired immune response activation in patients, which constitutes a major safety concern connected to the use of viral vectors in gene therapy applications in general and in cancer immunotherapy (i.e. CAR-T cell therapy) in particular. Moreover, in contrast to gamma-retroviral and lentiviral vectors that preferentially insert into actively transcribed or regulatory regions, SB presents a close-to-random genomic integration pattern reducing the risk of insertional mutagenesis and genotoxicity. Differently to genome editing nucleases as zinc-finger, TALENs, and Cas9, the SB transposase directly and precisely integrates its cargo into the chromosome without generating potentially harmful double-strand breaks at the target locus. Therefore, SB's insertion rates and safety do not depend on the efficiency of the repair machinery in the target cells. These advantages make SB the only non-viral gene delivery vehicle used for CAR-T cell engineering in clinical trials, which has triggered considerable commercial interest in the SB system in the last few years.

Although the SB system typically has lower gene-transfer efficiency than viral vectors, novel strategies-such as improved design and delivery methods for its components, as well as selective propagation of CAR positive cells—have recently increased the success of SB-mediated T cell engineering to levels similar to viral approaches. Despite these improvements, important issues remain. In particular, long-term transposase expression can result in uncontrolled ongoing transposition, potentially leading to transgene remobilization, undesired insertion events, genome instability and cytotoxicity. Moreover, insertion of the SB transposase gene from the expression vector (e.g. by homologous recombination) may result in infinite transposase production and unintended acquisition of the transposase promoter might cause activation of oncogenes or disrupt gene regulatory networks in the target cells. This poses concerns regarding the safety of the current SB system and highlights the need for technological advances to reduce or alleviate these risks.

To circumvent these risks, direct delivery of the SB transposase protein is highly desired, as it can help to achieve tighter efficiency/temporal control and improve the safety of transposition-based cell engineering, especially for therapeutic applications. Nevertheless, the production of active recombinant SB transposase in sufficient quantity and quality has been challenging to date.

The above problem is solved in a first aspect by a transposase polypeptide comprising at least one mutated amino acid residue compared to a reference amino acid sequence—such as a non-mutated but artificial transposase or a wild-type enzyme—of the transposase, wherein the at least one mutated amino acid residue is located within the catalytic domain of the transposase. The catalytic domain is preferably within an amino acid sequence between residues 150 and 250 of for example SB100x (SEQ ID NO: 2). Reference transposases according to the invention are preferably SB transposases either as wild-type enzymes or genetically engineered enzymes such as SB10, SB11 or SB100X.

The transposase polypeptide has several surprising advantages compared to the prior art SB100x enzyme. The invention shows in the example section that the transposase of the invention has a higher protein yield in recombinant protein expression, increased solubility, which is advantageous for delivery of the protein via electroporation (better soluble in the electroporation buffer), the enzyme is more stable and less prone to protein degradation and in particular more thermostable than the prior art enzyme which also is advantageous during electroporation.

The present invention provides a safe and effective strategy to achieve efficient, stable and controlled genetic engineering of mammalian cells via the direct delivery of a mutated transposase variant. The new transposase variant of the invention is suitable for large-scale recombinant protein production and transfection, which allows for successful engineering of a range of mammalian cell lines and the manufacture of Chimeric Antigen Receptor (CAR) T-cells. The present strategy, that was named “SBprotAct”, provides a novel approach to alleviate safety issues and enables maximal control of the transposase system in clinical applications. Finally, the transposase of the invention proved to create less insertions per cell at the same transgenesis rate and hence allows for a tightly controlled gene delivery.

The term “transposase” as used herein refers to an enzyme that is a component of a functional nucleic acid-protein complex capable of transposition and which is mediating transposition. The term “transposase” also refers to integrases from retrotransposons or of retroviral origin. A “transposition reaction” as used herein refers to a reaction where a transposon inserts into a target nucleic acid. Primary components in a transposition reaction are a transposon and a transposase or an integrase enzyme. For example, the transposase system according to the invention is preferably a so called “Sleeping Beauty (SB)” transposase. In certain aspects, the transposase is an engineered enzyme with improved characteristics such as increased enzymatic function. Some specific examples of an engineered SB transposases include, without limitation, SB10, SB11 or SB100x SB transposase (see, e.g., Mates et al., Nat. Gen. 2009, incorporated herein by reference). Other transposition systems can be used, e.g., Ty1 (Devine and Boeke, 1994, and WO 95/23875), Tn7 (Craig, 1996), Tn 10 and IS 10 (Kleckner et al. 1996), Mariner transposase (Lampe et al., 1996), Tc1 (Vos et al., 1996), Tn5 (Park et al., 1992), P element (Kaufman and Rio, 1992) and Tn3 (Ichikawa and Ohtsubo, 1990), bacterial insertion sequences (Ohtsubo and Sekine, 1996), retroviruses (Varmus and Brown 1989), and retrotransposon of yeast (Boeke, 1989).

In preferred embodiments of the present invention the transposase is a Sleeping Beauty (SB) transposase, and preferably is SB100X (SEQ ID NO: 2) or an enzyme derived from SB100X.

Hence, the transposase polypeptide according to the invention is a polypeptide having transposase activity, wherein the at least one mutated amino acid residue is a residue that is located between amino acid 150 and 250 of SB transposase, preferably SB100X transposase.

In some embodiments it is preferably that the at least one mutated amino acid residue is at least two mutated amino acid residues, or at least three, four, five or more amino acids. It is preferably that the transposase polypeptide of the invention when its sequence is aligned with the sequence of an SB transposase, preferably SB100X, is mutated in any one of amino acids 170 to 180 and/or 207 to 217. More preferably the at least one mutated amino acid residue is selected from amino acid 176 and/or 212 of SB transposase, preferably of SB100X. Most preferably the at least one mutated amino acid residue is mutated into a serine residue, and preferably is C176S, or C176S and I212S.

In other embodiments, the transposase polypeptide of the invention further comprises an amino acid sequence having at least 60%, 70%, 80%, 90%, 95%, 96%, 97%, 98%, 99%, most preferably 100% sequence identity to the amino acid sequence between residues 150 to 250 as shown in SEQ ID NO: 1 (hsSB). It is preferred that the transposase polypeptide includes at least a C176 mutation, preferably C176S, compared to the sequence in SEQ ID NO: 2. Even more preferably the transposase polypeptide further includes the mutation at position I212, preferably I212S.

In some embodiments the transposase polypeptide of the invention comprises an amino acid sequence having at least 60%, 70%, 80%, 90%, 95%, 96%, 97%, 98%, 99%, most preferably 100% sequence identity to the full length amino acid sequence as shown in SEQ ID NO: 1 or 3 (hsSB). Preferably, although the degree of sequence identity is in some embodiments below 100%, the above indicated at least one mutation shall be present in the transposase polypeptide of the invention.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of the transposase of the invention to for example a reference (non-mutated transposase), the percentage identity can be determined by the Blast searches provided in NCBI; in particular for amino acid identity, those using BLASTP 2.2.28+ with the following parameters: Matrix: BLOSUM62; Gap Penalties: Existence: 11, Extension: 1; Neighboring words threshold: 11; Window for multiple hits: 40.

In addition, in some embodiments, the transposase polypeptide of the invention has an increased solubility compared to a reference non-mutated transposase polypeptide, preferably wherein the reference non-mutated transposase polypeptide is SB100X transposase, preferably as shown in SEQ ID NO: 2 (non-mutated SB100X).

In another aspect of the invention there is provided a polynucleotide comprising a nucleic acid sequence encoding for a transposase polypeptide as described herein above, preferably wherein the polynucleotide is RNA or DNA. For example RNA may be provided in the form of messenger RNA (mRNA) that allows for a direct translation into the transposase polypeptide of the invention if the mRNA is introduced into a biological cell.

Another aspect of the invention pertains to a vector comprising a polynucleotide of the invention. Also provided is an expression construct, comprising an expressible polynucleotide encoding a transposase polypeptide of the invention and a promoter element, wherein the promoter element is operably linked to the expressible polynucleotide to allow for the expression of the polynucleotide.

Also provided is a recombinant cell, comprising a transposase polypeptide of the invention, a polynucleotide of the invention, or a vector and/or an expression construct of the invention.

The recombinant cell is preferably a cell suitable for recombinant protein expression, preferably for recombinant protein expression of the transposase polypeptide of the invention. Such as a bacterial cell or eukaryotic cell, most preferably a bacterial cell such as E. coli or an insect cell, such as Drosophila S2 cell or a mammalian cell such as HEK293T cell.

Yet another aspect relates to a transposon system comprising
(a) a transposon unit containing inverted terminal repeats (ITRs) or direct terminal repeats (DTRs) that flank a sequence of interest to be inserted into the genome of a target cell; and
(b) a transposase polypeptide, a polynucleotide, a vector and/or an expression construct as described herein above.

Herein, the term “transposon unit” shall refer to the nucleic acid construct that constitutes the transposon genetic sequence with the target sequence that is to be introduced into a target cell genome. Usually a transposon unit will be nucleic acid and may be a vector of any form suitable for transposition.

As used herein, the term “inverted terminal repeat” refers to a sequence located at one end of a transposon unit that can be cleaved by a transposase polypeptide when used in combination with a complementary sequence that is located at the opposing end of the vector or transposon unit. The pair of inverted terminal repeats is involved in the transposition activity of the transposon of the transposon unit of the present disclosure, in particular involved in DNA addition or removal and excision and integration of DNA of interest. In one example, at least one pair of an inverted terminal repeat appears to be the minimum sequence required for transposition activity in a plasmid. In another example, the transposon unit of the present disclosure may comprise at least two, three or four pairs of inverted terminal repeats. As would be understood by the person skilled in the art, to facilitate ease of cloning, the necessary terminal sequence may be as short as possible and thus contain as little inverted repeats as possible. Thus, in one example, the transposon unit of the present disclosure may comprise not more than one, not more than two, not more than three or not more than four pairs of inverted terminal repeats. In one example, the transposon unit of the present disclosure may comprise only one inverted terminal repeat. Whilst not wishing to be bound by theory, it is envisaged that having more than one inverted terminal repeat may be disadvantageous as it may lead to non-specific transposase binding to the multiple inverted terminal repeats and resulting in the removal of desired sequence or insertion of undesirable sequences. The inverted terminal repeat of the present disclosure may form either a perfect inverted terminal repeat (or interchangeably referred to as “perfect inverted repeat”) or imperfect inverted terminal repeat (or interchangeably referred to as “imperfect inverted repeat”). As used herein, the term “perfect inverted repeat” refers to two identical DNA sequences placed at opposite direction. The above descriptions for transposon units with ITR also apply for transposon units with DTRs.

A transposon system that could be used with the inventive transposon polypeptide/nucleic acid of the invention is for example disclosed in WO 2017/050448 A1, which is included in the present disclosure by reference.

A transposon system according to the invention is preferable, wherein said transposon unit of (a) is in the form of a minicircle. However, the transposon unit may be other nucleic acid systems. However, minicircles are preferable in the context of T cell engineering.

The transposon system of the invention in preferred embodiments is an SB transposon system.

Another aspect of the invention then pertains to the use of a transposon system as described for gene delivery into a target cell. The gene delivery is preferably an ex vivo gene delivery into a target cell, such as a target cell selected from a stem cell, such as a hematopoietic or embryonic stem cell, T-cell, B-Cell or Chinese Hamster Ovary (CHO) cell. Most preferably is the system or the compounds of the invention used in the generation of CAR T-cells.

The term “chimeric antigen receptor” (CAR), as used herein, refers to an extracellular antigen-binding domain that is fused to an intracellular signaling domain of a cell, such as a T cell or a NK-92 cell.

Further provided is a method for gene delivery into a target cell comprising the following steps:
(a) bringing into contact the transposon system as described with a target cell;
(b) culturing said target cell under conditions permissive to the culture of said target cell.

In another aspect, a pharmaceutical composition is provided, comprising a transposase polypeptide, a polynucleotide, a vector, and/or an expression construct, together with a pharmaceutically acceptable carrier and/or excipient.

Another aspect then pertains to a kit comprising
(a) a transposon unit containing inverted terminal repeats (ITRs) or DTRs that flank a sequence of interest to be inserted into the genome of a target cell; and
(b) a transposase polypeptide, a polynucleotide, a vector, and/or an expression construct of the invention as described herein.

The compounds and systems of the invention may preferably find application in medicine. Therefore, such compounds and systems of the invention are preferably for use in the treatment of a disease. Such diseases may be proliferative disease, such as cancer. For a cancer treatment, the invention may be used in context of the generation of modified immune cells. For example, the invention can be used to introduce into immune cells T cell receptors (TCR) or CARs or other immune molecules, to strengthen and target a patient's immune system against cancer cells. Immune cells that can be modified may be selected from human T lymphocytes or B cells. Other diseases that could benefit from the invention are genetic disorders that are characterized by the loss of a gene function. In such diseases cells could be modified with the invention to include a healthy copy of the disease associated gene. Other target cells that are preferably used in context with the invention are stem cells such as, for example, embryonic, or adult stem cells, such as hematopoietic stem cells.

The following figures, sequences, and examples merely serve to illustrate the invention and should not be construed to restrict the scope of the invention to the particular embodiments of the invention described in the examples. All references as cited herein are hereby incorporated in their entirety by reference.

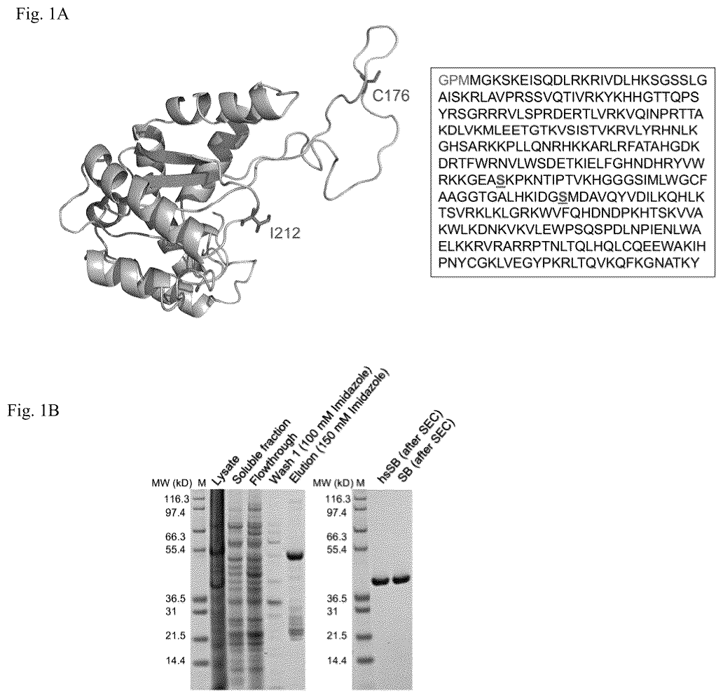

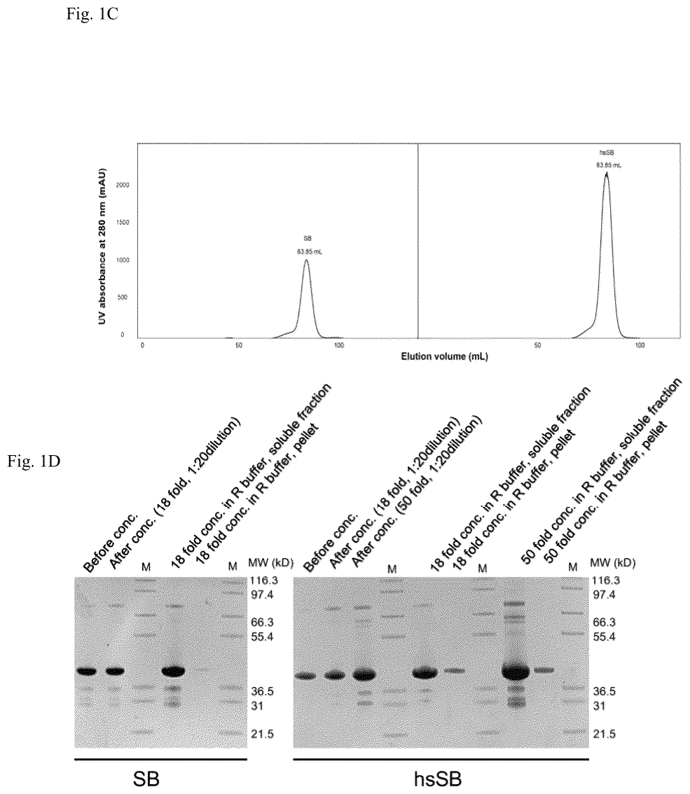

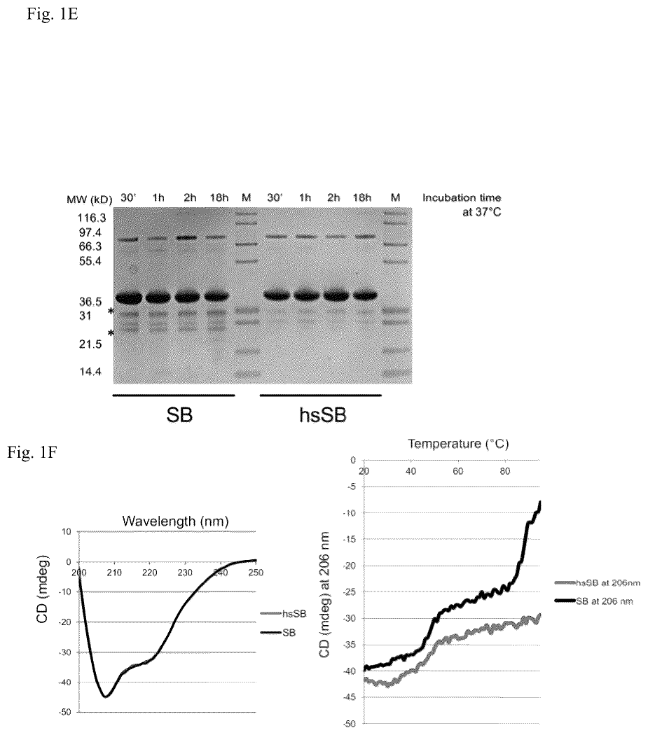

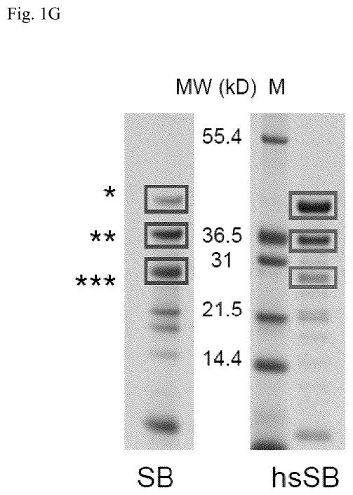

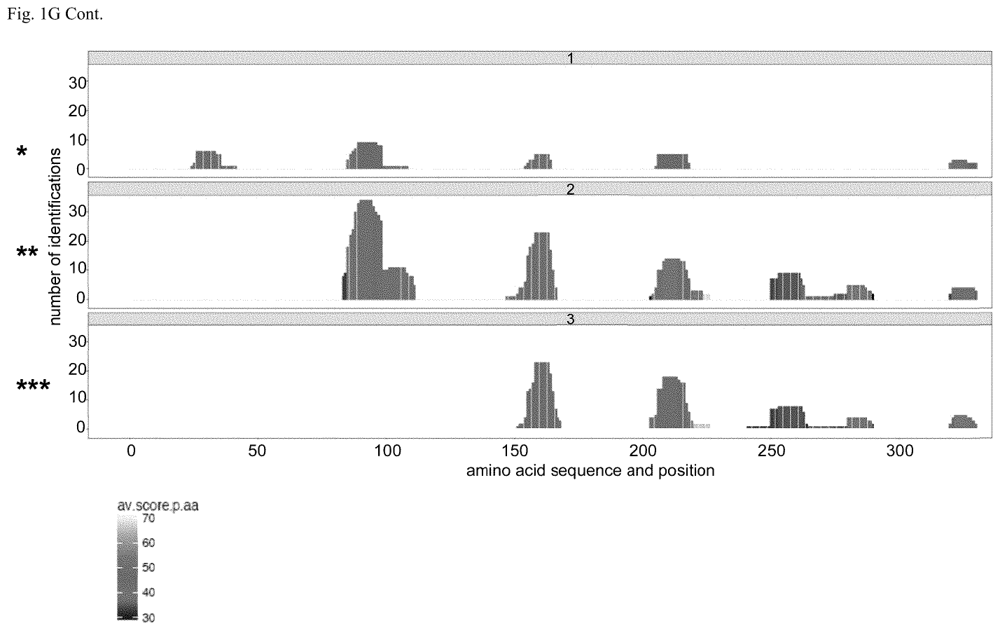

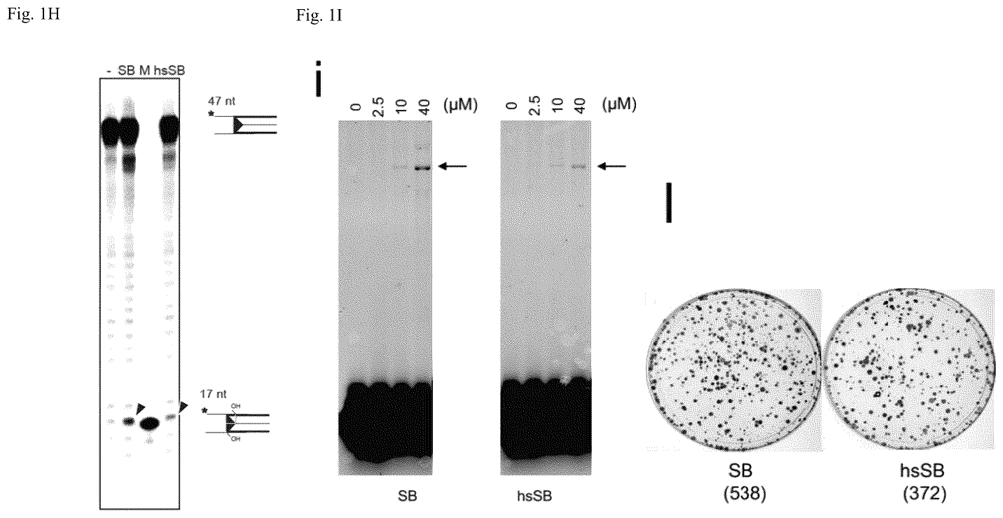

FIGS. 1A-1I: Rational mutagenesis of the SB100X transposase

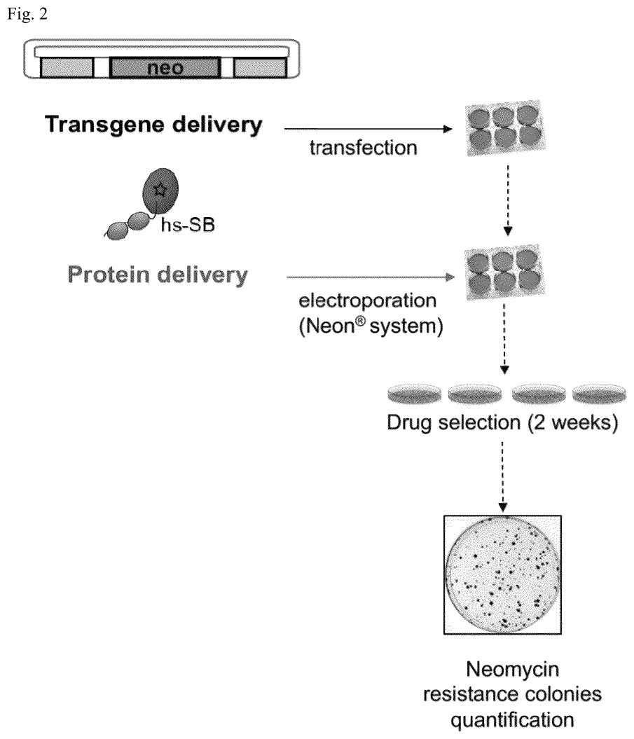

FIG. 2: Schematic representation of the SBprotAct engineering procedure.

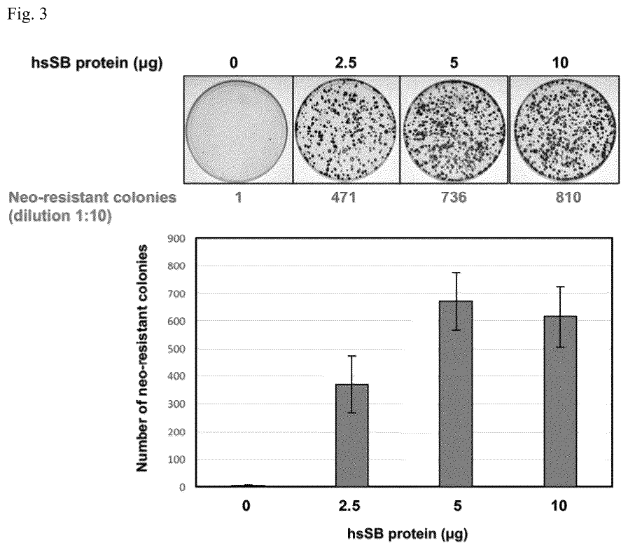

FIG. 3: Transgene (neomycin) insertions are driven by transposition activity of the transfected hsSB transposase.

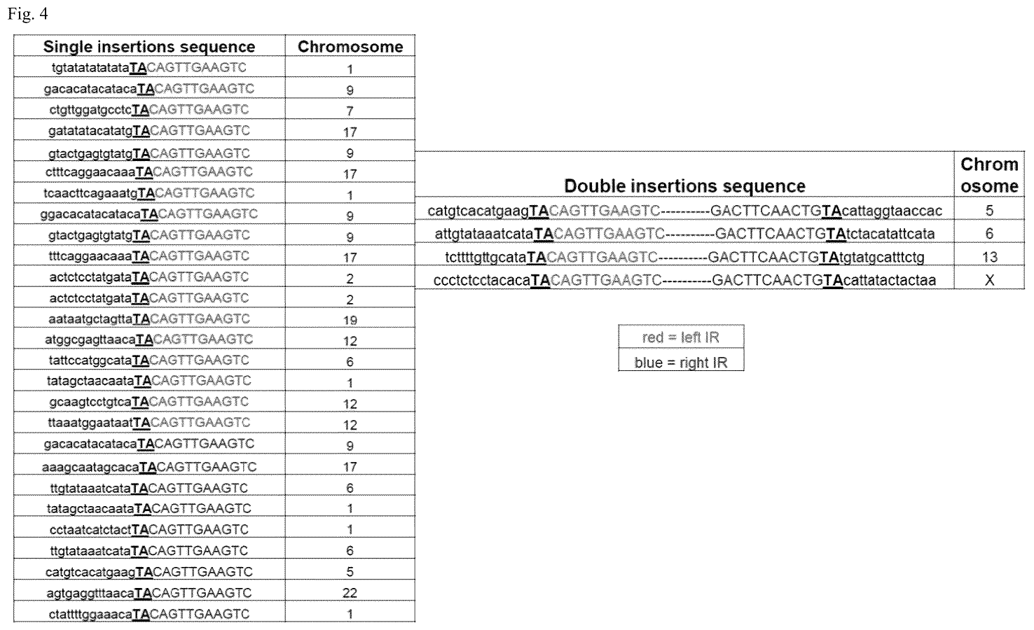

FIG. 4: Insertion sites as derived by sequence analysis of the neomycin locus from 11 isolated neomycin positive HeLa cell clones. Insertions of SB IRs correctly occur at TA dinucleotides.

FIGS. 5A-5E: Delivery of hsSB by protein or plasmid. FIG. 5A: retention of hsSB delivered into HeLa cells as protein or expressed from plasmid DNA; FIG. 5B and FIG. 5C: Representative transposition assays in Hela cells demonstrate comparable transgene (neomycin resistance) insertion rate when using the hsSB transposase delivered as a plasmid (FIG. 5B) or as a protein (FIG. 5C). Error bars indicate the standard error of the mean from 2 independent experiments (n=2); FIGS. 5D and 5E: representative flow cytometric analysis by Fluorescence Activated Cell Sorting (FACS) of HeLa cells transfected with Venus-carrying transposon plasmid and: (FIG. 5D) hsSB plasmid, or (5E) hsSB delivery by electroporation. Cells that acquired the transposon plasmid were sorted 2 days post-transfection; transposition efficiency was quantified 21 days later by FACS analysis.

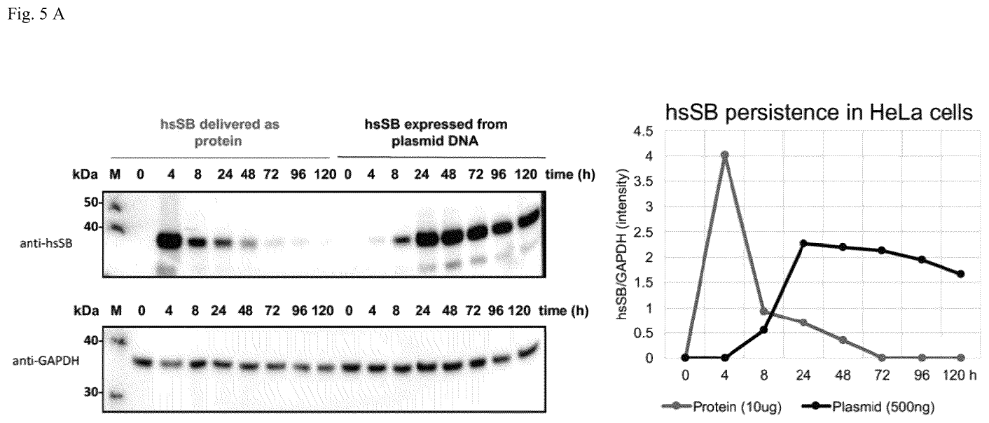

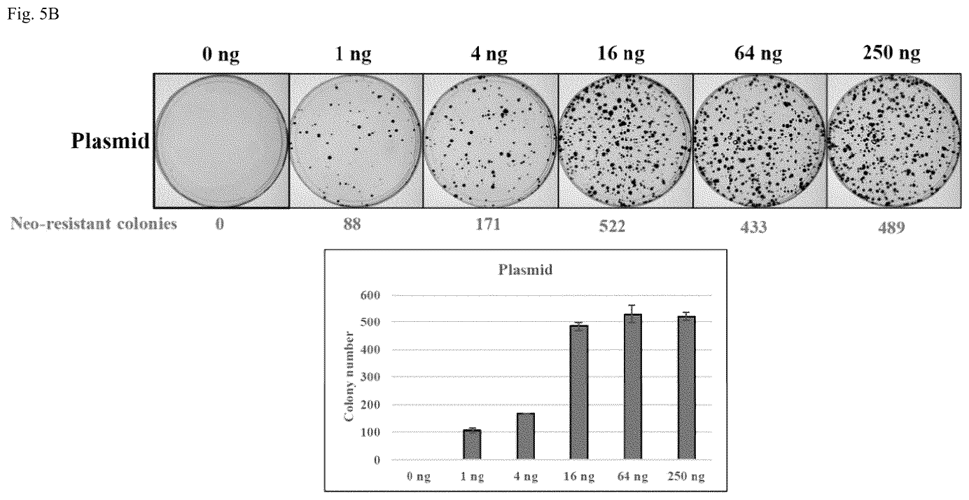

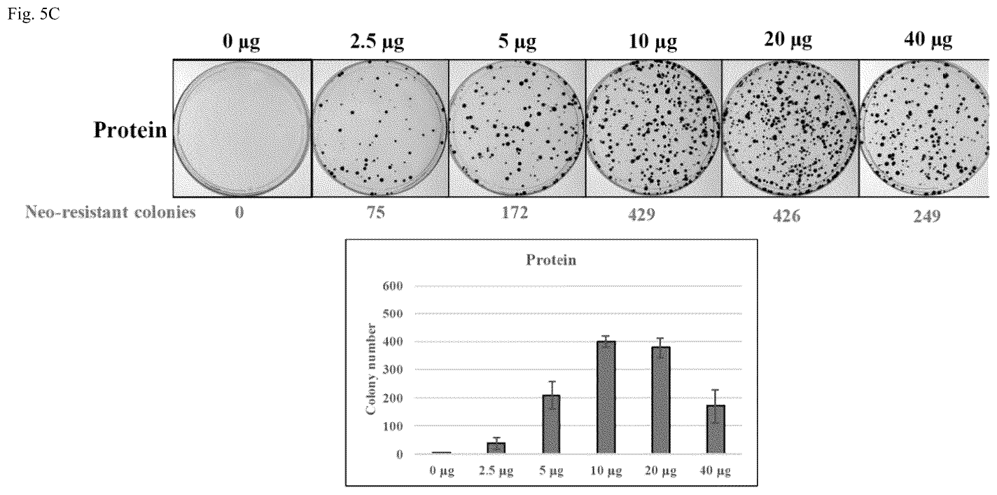

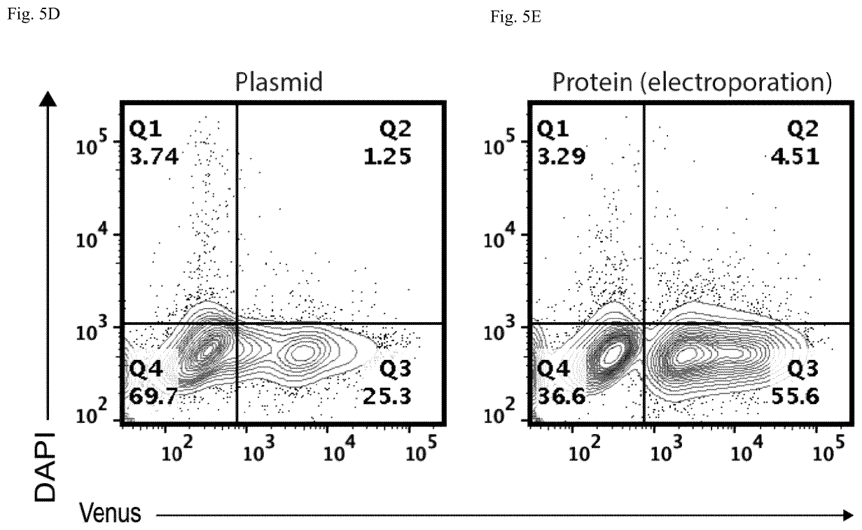

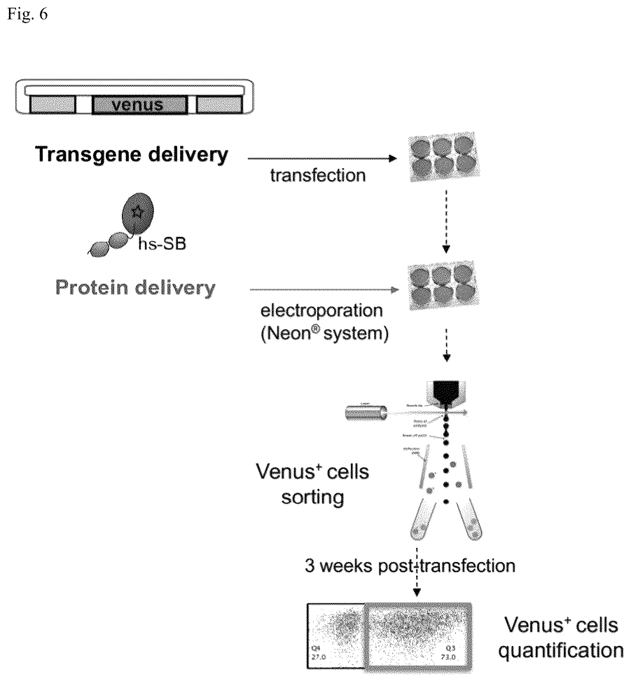

FIG. 6: Schematic representation of the SBprotAct engineering procedure with quantification by cell sorting.

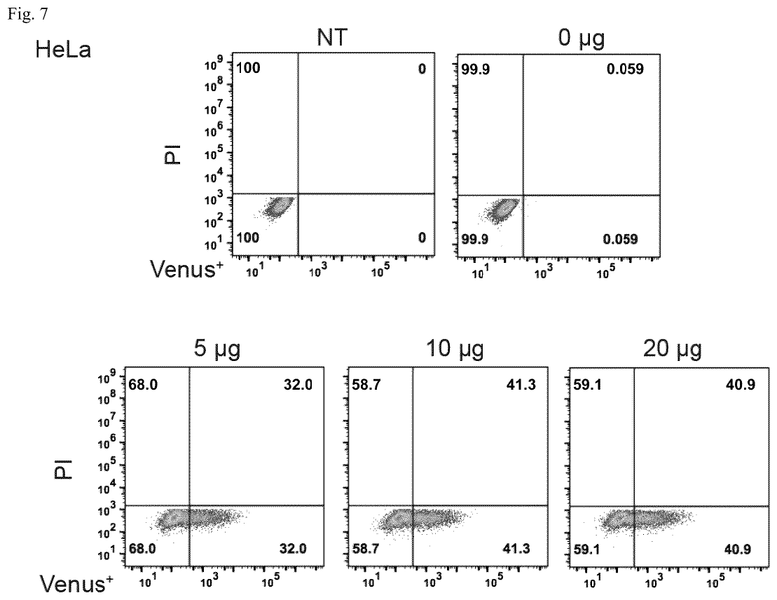

FIG. 7: Representative flow cytometric analysis of HeLa cells transfected with Venus-carrying transposon plasmid and electroporated with hsSB transposase.

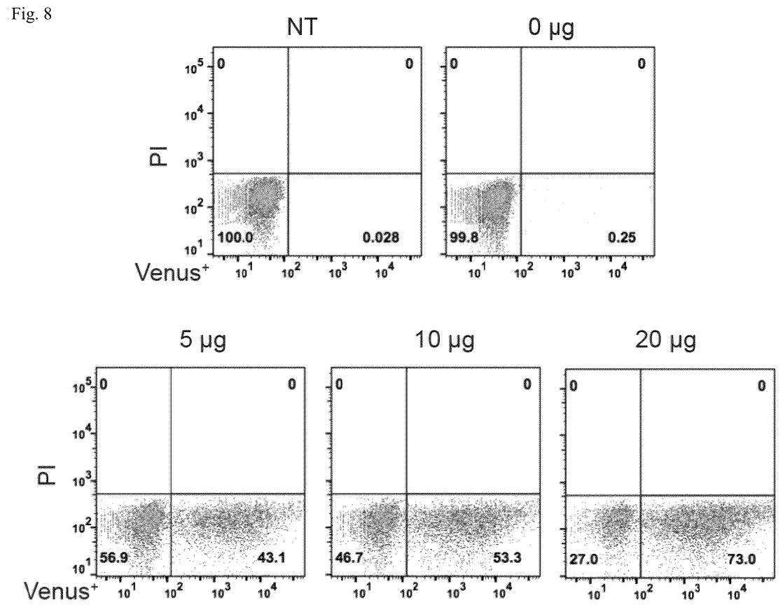

FIG. 8: Representative flow cytometric analysis of Chinese Hamster Ovary (CHO) cells transfected with Venus-carrying transposon plasmid and electroporated with hsSB transposase.

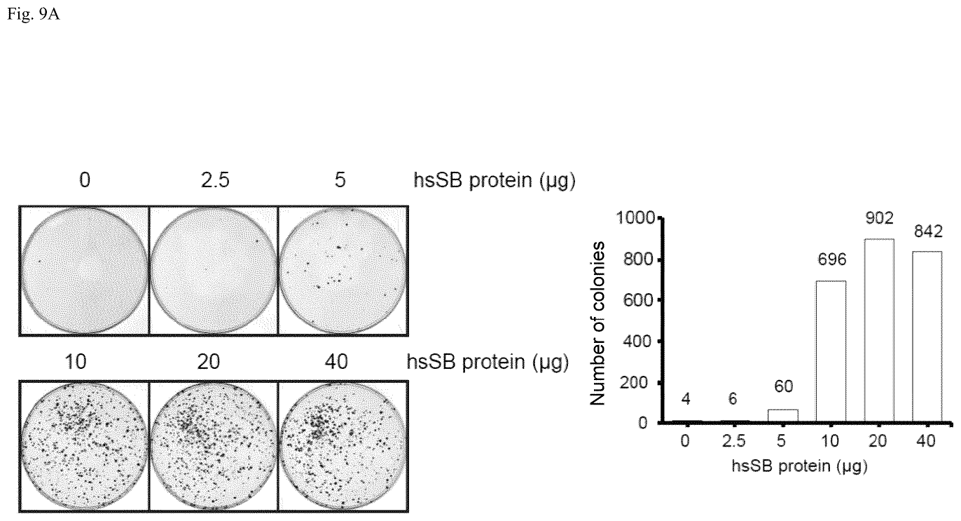

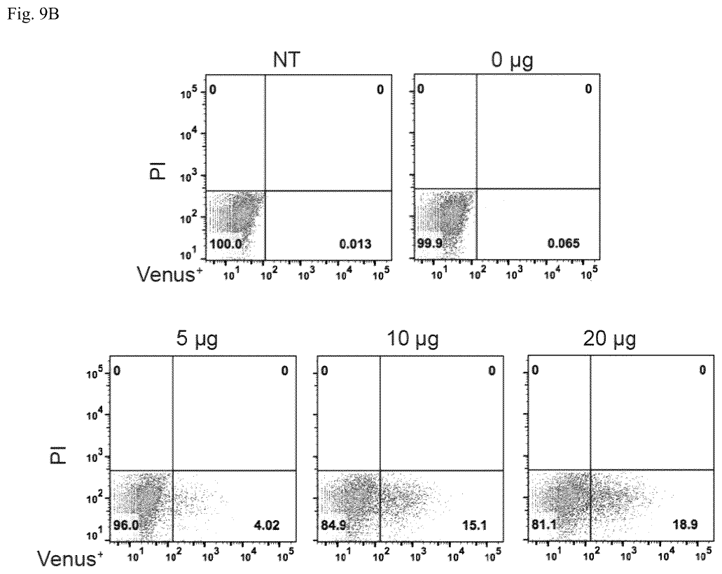

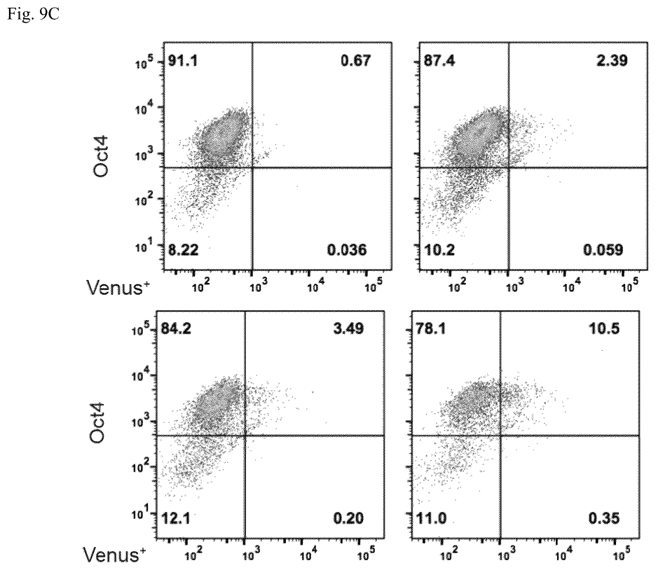

FIGS. 9A-9C: Genetic engineering of mESCs by direct delivery of the hsSB transposase protein.

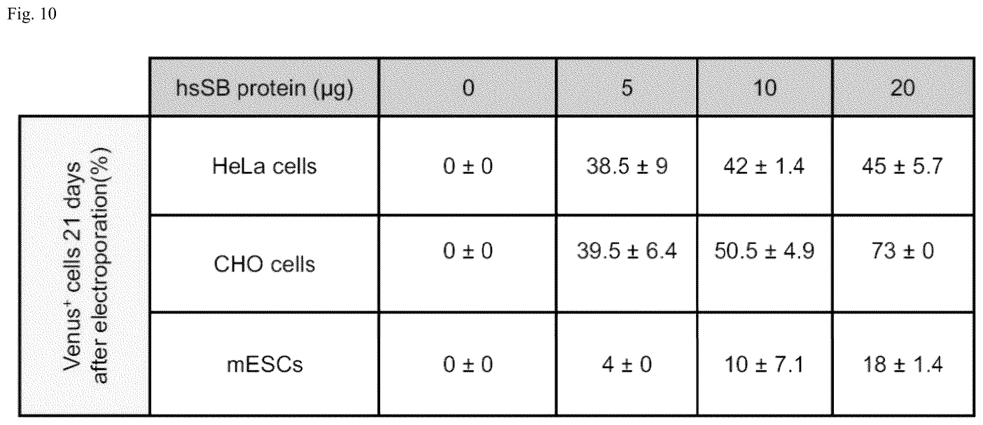

FIG. 10: Transgenesis efficiency of the SBprotAct system in different cell lines as quantified by flow cytometric analysis. Errors are indicated as standard deviation (n=2).

(hsSB)
SEQ ID NO: 1
MGKSKEISQDLRKRIVDLHKSGSSLGAISKRLAVPRSSVQTIVRKYKHHG
TTQPSYRSGRRRVLSPRDERTLVRKVQINPRTTAKDLVKMLEETGTKVSI
STVKRVLYRHNLKGHSARKKPLLQNRHKKARLRFATAHGDKDRTFWRNVL
WSDETKIELFGHNDHRYVWRKKGEASKPKNTIPTVKHGGGSIMLWGCFAA
GGTGALHKIDGSMDAVQYVDILKQHLKTSVRKLKLGRKWVFQHDNDPKHT
SKVVAKWLKDNKVKVLEWPSQSPDLNPIENLWAELKKRVRARRPTNLTQL
HQLCQEEWAKIHPNYCGKLVEGYPKRLTQVKQFKGNATKY
(non mutated SB100X)
SEQ ID NO: 2
MGKSKEISQDLRKRIVDLHKSGSSLGAISKRLAVPRSSVQTIVRKYKHHG
TTQPSYRSGRRRVLSPRDERTLVRKVQINPRTTAKDLVKMLEETGTKVSI
STVKRVLYRHNLKGHSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMDAVQYVDILKQHLKTSVRKLKLGRKWVFQHDNDPKHT
SKVVAKWLKDNKVKVLEWPSQSPDLNPIENLWAELKKRVRARRPTNLTQL
HQLCQEEWAKIHPNYCGKLVEGYPKRLTQVKQFKGNATKY
(hsSB for recombinant expression)
SEQ ID NO: 3
GPMMGKSKEISQDLRKRIVDLHKSGSSLGAISKRLAVPRSSVQTIVRKYK
HHGTTQPSYRSGRRRVLSPRDERTLVRKVQINPRTTAKDLVKMLEETGTK
VSISTVKRVLYRHNLKGHSARKKPLLQNRHKKARLRFATAHGDKDRTFWR
NVLWSDETKIELFGHNDHRYVWRKKGEASKPKNTIPTVKHGGGSIMLWGC
FAAGGTGALHKIDGSMDAVQYVDILKQHLKTSVRKLKLGRKWVFQHDNDP
KHTSKVVAKWLKDNKVKVLEWPSQSPDLNPIENLWAELKKRVRARRPTNL
TQLHQLCQEEWAKIHPNYCGKLVEGYPKRLTQVKQFKGNATKY

(underlined are mutated or to-be mutated residues. Bold and italic are residues introduced for recombinant protein expression)

### EXAMPLES

### Example 1: Rational Mutagenesis of the SB100X Transposase

In FIG. 1A, left side the crystal structure of the SB100X transposase (hereafter referred to as SB) catalytic domain is shown. Residues mutated to serines for the generation of the hsSB variant are shown as sticks in magenta. Right: Amino acid sequence of the full length hsSB transposase variant used for recombinant protein production. Bold underlined characters in magenta indicate serines substituting C176 and 1212 respectively in the SB100X sequence. Residues colored in blue have been introduced at the N-terminus for recombinant protein production.

FIG. 1B shows the recombinant production of hsSB protein of the invention. SDS-PAGE analysis of purified hsSB protein variant is provided. hsSB is recombinantly produced in E. coli (fused to N-terminal purification and solubility tags) in high quantity. hsSB is highly pure after tag removal and size exclusion chromatography (SEC). Purification yields of hsSB are shown in FIG. 1Ce. Size exclusion chromatogram showing that hsSB is recombinantly produced at significantly higher yields (roughly double amounts) compared to SB, indicating improved solubility of the hsSB variant. High solubility of hsSB in electroporation buffer is shown in FIG. 1D. hsSB can be concentrated up to 50 fold (corresponding to 20 mg/ml), whereas SB undergoes precipitation at concentrations higher than 7 mg/ml. hsSB is highly soluble in the low salt R buffer (used for electroporation), even at high protein concentration. While some precipitation is observed upon concentration, the vast majority of hsSB stays in the soluble fraction. SDS-PAGE analysis of purified SB proteins upon incubation at 37° C. is shown in FIG. 1Ee. SB exhibits degradation (degradation products indicated by asterisks) even upon short incubation at physiological temperature, while hsSB does not. Also, as shown in FIG. 1F, hsSB is more thermostable than SB. CD measurements of both proteins in close to physiological (200 mM NaCl, pH 7.5) buffer condition. However, hsSB has the same fold as SB, as shown in the left panel. Nonetheless, hsSB is significantly more thermostable; it still does not completely unfold at 95° C. (right panel). Considering that electroporation heats the sample, this property is highly advantageous for protein transfection. Finally, upon long-term storage hsSB is better preserved than SB (FIG. 1g). Left panel: SDS-PAGE analysis of purified SB proteins upon long-term storage at −80° C., showing that SB undergoes significant degradation after freezing, while hsSB does not. Right panel. Mass spectrometry analysis of the bands indicated by blue boxes and asterisks on the left confirms that the bands correspond to degradation products of the SB protein.

### Example 2: Gene Delivery Using hsSB Transposase into Hela Cells

A strategy for gene delivery is depicted in FIG. 2. Transgene (neomycin resistance gene) insertions are driven by transposition activity of the transfected hsSB transposase (FIG. 3). Top: Representative transposition assay in Hela cells. Number of neomycin resistant colonies is shown in parenthesis. Bottom: Quantification of the transposition assay in HeLa cells. Error bars represent standard error (n=3).

FIG. 4 shows insertion sites as derived by sequence analysis of the neomycin locus from isolated neomycin positive HeLa cells. Insertions of SB IRs correctly occur at TA dinucleotides.

FIG. 5A shows the retention of hsSB delivered into HeLa cells as protein or expressed from plasmid DNA. Western blot analysis shows almost complete loss of delivered hsSB protein 48 hours after electroporation, whereas cells transfected with hsSB expression plasmids produce high level of protein continuously from 24 hours to 5 days after transfection. Western blot was performed on lysate from Hela cells transfected with 0.5 μg of pSBTer (Tpn) and electroporated with 10 μg hsSB protein or transfected with 0.5 μg hsSB expression plasmid. Samples were taken at the indicated time points and 20 μg-s of the total lysate were separated by electrophoresis and transferred to a nitrocellulose membrane. The SB was detected with anti-SB antibody. The internal loading control was glyceraldehyde 3-phosphate dehydrogenase (GAPDH) detected with anti-GAPDH antibody. Measurement of the intensities of the bands allows to quantify hsSB persistence in Hela cells over time (as shown in the chart on the left).

A Comparison of genetic engineering efficiency in Hela cells as executed by hsSB provided on an expression plasmid or directly delivered as a protein by electroporation is shown in FIG. 5B to 5E. As can be seen from the figures, genetic engineering efficiency does not depend on the transfection method, although protein electroporation is for a shorter time period more efficient, whereas plasmid transfection yields long term expression of the transposase.

A schematic representation of the SBprotAct engineering procedure with quantification by cell sorting is shown in FIG. 6. FIG. 7 shows a representative flow cytometric analysis of HeLa cells transfected with Venus-carrying transposon plasmid and electroporated with hsSB transposase. Venus-positive cells are identified 3 weeks post-transfection, so as to select for transposition positive cells. The electroporated hsSB protein amounts are indicated above each chart. Y axis: propidium iodide (PI) staining to select living cells. X-axis: green fluorescence from Venus. NT: non-transfected.

### Example 3: Gene Delivery Using hsSB Transposase in CHO Cells and Mouse Embryonic Stem Cells (mESCs)

A representative flow cytometric analysis of Chinese Hamster Ovary (CHO) cells transfected with Venus-carrying transposon plasmid and electroporated with hsSB transposase is shown in FIG. 8. Venus-positive cells are identified 3 weeks post-transfection, so as to select for transposition positive cells. The electroporated hsSB protein amounts are indicated above each chart. Y axis: propidium iodide (PI) staining to select living cells. X-axis: green fluorescence from Venus. NT: non-transfected.

FIGS. 9A-C show genetic engineering of mESCs by direct delivery of the hsSB transposase protein. FIG. 9A: Representative transposition assay in mouse embryonic stem cells (mESCs) demonstrating efficient transgene (neomycin resistance) insertions by the transfected hsSB transposase. FIG. 9B: Representative flow cytometric analysis of mESCs transfected with Venus-carrying transposon plasmid and electroporated with hsSB transposase. Venus-positive cells are identified 3 weeks post-transfection, so as to select for transposition positive cells. The electroporated hsSB protein amounts are indicated above each chart. Y axis: propidium iodide (PI) staining to select living cells. X-axis: green fluorescence from Venus. NT: nontransfected. FIG. 9C: Oct4 staining confirms that engineered mESCs retain their pluripotent state.

Transgenesis efficiency of the SBprotAct system in different cell lines is shown in FIG. 10 as quantified by flow cytometric analysis. Errors are indicated as standard deviation (n=2).

FIG. 11. (a) shows a representative transposition assay in mouse embryonic stem cells (mESCs) demonstrating efficient transgene (neomycin) insertions by the transfected hsSB transposase. In (b) a representative flow cytometric analysis of mESCs transfected with Venus carrying transposon plasmid and electroporated with hsSB transposase is provided. Venus-positive cells are identified 3 weeks post-transfection, so as to select for transposition positive cells. The electroporated hsSB protein amounts are indicated above each chart. Y axis: propidium iodide (PI) staining for living cells. X-axis: green fluorescence from Venus. NT: nontransfected. In FIG. 11 (c) Oct4 staining confirms that engineered mESCs retain their pluripotent state.

In a further experiment the inventors sought to quantify transposition efficiency. Transgenesis efficiency of the SBprotAct system in different cell lines was quantified by flow cytometric analysis. Results are provided in FIG. 12, errors are indicated as standard deviation (n=2).

In conclusion, the novel transposase variant and transfection strategy (i.e. SBprotAct) establishes a new generation of the SB transposon system for cell engineering based on the use of purified transposase protein, which is unprecedented in itself to date. In standard SB-based applications, expression of the SB transposase is achieved either from an expression plasmid or from protein-encoding messenger RNA delivered into target cells. In ongoing clinical gene therapy trials, expression plasmids are exclusively used as sources of the SB transposase. In comparison to transposase gene delivery, direct hsSB protein delivery in SBprotAct provides:
a) Comparable transgenesis rates in diverse cell types.
b) No risks of transposase-gene or -promoter integration, circumventing uncontrolled long-term transposition and undesired transcriptional activation (of e.g. oncogenes) in the target cells.
c) No need for transcription and translation in the target cells. This expands the applicability of SB-mediated engineering to cells in which protein over-expression is difficult and/or compromises cell viability.
d) Fast cell engineering and rapid protein turnover, as hsSB protein is degraded within 48 h from delivery. Therefore hsSB protein acts in a hit-and-run fashion, minimizing off-target activities (see below).
e) Lower cytotoxicity, reduced risks of insertional mutagenesis and transgene remobilization due to limited temporal window of transposition.
f) Lower number of insertions per cell at the same transgenesis rate, minimizing genome perturbations.
g) Dose-dependent efficiency. By varying the concentration of the transfected hsSB protein, the number of positive clones can be tightly controlled.
h) Discrete and adjustable number of insertion events. hsSB-mediated engineering produces clones with discrete number of insertions per genome, which can be adjusted by varying the protein dose. In contrast, uncontrolled level and time of transposition from expression plasmids leads to heterogeneous, multicopy clones.

In addition, for CAR-T cell generation the use of mRNA has been explored as alternative source of SB transposase, but the instability of mRNA raises quality control issues that could hinder widespread use for therapy. hsSB protein delivery offers several advantages in comparison to mRNA delivery:
i) Independence from cellular translation efficiency and regulation.
j) Even tighter and more direct control of transposition efficiency, since the SB transposase immediately works after transfection without the need for translation.
k) The possibility to assess protein quality and activity in vitro prior to application (assays described in publication and shown in FIG. 1h herein). This is of particular relevance for quality control procedures in a commercial or clinical setting.

To date, delivery of active transposases for high-efficient mammalian cell engineering has been achieved only for the PiggyBac transposase, but required incorporation of the protein into lentiviral particles. Thus, SBprotAct provides for the first time a completely virus-free system for efficient delivery of a transposase protein in a medically relevant setting, avoiding all safety concerns and financial limitations connected to the use and the manufacture of viral vectors.

In summary, our invention, the SBprotAct system, opens up new possibilities to achieve maximal control of SB transposition in its genetic engineering applications, making SB an ever-safer genetic tool. Direct delivery of the hsSB protein allows rapid transposase clearance from the cell, avoiding the undesired effects of long-term transposition. Moreover, providing directly the active factor of transposition, the rates and time frame of active transgene insertion can be finely modulated and do not depend on the timeline and stochastic events in transposase expression (from plasmids) or translation (from mRNAs) by the cellular machinery, thereby also avoiding the fitness costs for the target cell.

Presently, SB is the only non-viral gene delivery tool currently used to manufacture CAR T-cells in clinical trials and it has already advanced quite far in clinical development. While preserving all advantages of the current SB system, including simplicity, ease and low cost, the SBprotAct of the invention provides a novel approach to overcome safety issues concerning the use of the current SB system in clinical applications.

1. An improved method for inserting a transgene into the genome of a living host cell;
wherein the method comprises introducing both a transposon that contains the transgene and a polynucleotide vector that encodes a Sleeping Beauty transposase into the host cell; and culturing the cell under conditions wherein the transposase is expressed in the cell and catalyzes insertion of the transgene into the cell's genome;
wherein the improved method comprises the following:
(a) identifying a mutated transposase that is at least 90% identical to the amino acid sequence of Sleeping Beauty transposase SB100X shown in SEQ. ID NO:2 and contains a plurality of amino acid mutations that increase solubility of the mutated transposase in relation to SB100X while maintaining transposase activity;
(b) obtaining said mutated transposase as an isolated protein; and
(c) introducing an effective amount of the mutated transposase protein into the host cell with said isolated protein in place of said polynucleotide vector, thereby catalyzing insertion of the transgene into the cell's genome.

2. The method of claim 1, wherein the mutated transposase is identified in step (a) by a process that comprises:
(i) producing a plurality of mutated transposase candidates, each of which is at least 90% identical to the amino acid sequence of Sleeping Beauty transposase SB100X shown in SEQ. ID NO:2 and contains a plurality of amino acid mutations between amino acid 150 and 250;
(ii) determining solubility of each of the candidates;
(iii) introducing each of the candidates into a host cell along with a transposon containing a reporter gene; and
(iv) determining insertion of the reporter gene into each of the host cells as an indication of transposase activity; thereby identifying one of the candidates as having increased solubility relative to SB100X while maintaining transposase activity.

3. The method of claim 2, wherein the candidates are designed by rational mutagenesis with reference to the crystal structure of SB100X.

4. The method of claim 2, wherein solubility of each candidate is determined in a low salt electroporation buffer in relation to solubility of SB100X.

5. The method of claim 2, wherein the reporter gene is a drug resistance gene, and transposase activity is determined by counting colonies of cells that survive after culturing in a medium that contains the drug.

6. The method of claim 2, wherein the reporter gene encodes a fluorescent protein, and transposase activity is determined by fluorescence-activated cell sorting (FACS).

7. The method of claim 1, wherein the mutated transposase is obtained as an isolated protein in step (b) by expressing a polynucleotide encoding the mutated transposase; and purifying protein obtained therefrom.

8. The method of claim 3, wherein at least one of the mutations in the mutated transposase identified in step (a) removes a polar amino acid in or around the DNA binding site of the crystal structure.

9. The method of claim 3, wherein at least one of the mutations in the mutated transposase identified in step (a) removes a cysteine on the surface of the crystal structure.

10. The method of claim 1, wherein the mutated transcriptase is soluble in a low salt electroporation buffer at concentrations above 7 mg/mL.

11. The method of claim 1, wherein the mutated transposase is more thermostable upon freezing and during storage at −80° C. compared with SB100X.

12. The method of claim 1, wherein the mutated transposase has higher transposase activity than SB100X.

13. The method of claim 1, wherein the mutated transposase protein inserted into a host cell has transposase activity that is comparable to transposase activity expressed in the host cell from a polynucleotide vector.

14. An improved method for inserting a transgene into the genome of a living host cell, comprising:
(a) obtaining a mutated transposase in protein form,
wherein the mutated transposase is at least 90% identical to the amino acid sequence of Sleeping Beauty transposase SB100X shown in SEQ. ID NO:2 and contains a plurality of amino acid mutations relative to SEQ. ID NO:2 that increase solubility of the mutated transposase relative to SB100X while maintaining transposase activity,
wherein at least one mutated amino acids is between amino acids 170 to 180 of SEQ. ID NO:2, and at least one mutated amino acid is between amino acids 207 to 217 of SEQ. ID NO:2;
(b) introducing an effective amount of the mutated transposase protein into the host cell in combination with a transposon that contains the transgene; and
(c) culturing the cell under conditions wherein the mutated transposase protein catalyzes insertion of the transgene into the genome of the host cell.

15. The method of claim 14, where said amino acid mutations replace one or more polar amino acids or cysteine residues in SEQ. ID NO:2 with a non-polar amino acid.

16. The method of claim 14, wherein at least one of said amino acid mutations is at position 176 of SEQ. ID NO:2.

17. The method of claim 14, wherein least one of said amino acid mutations is at position 212 of SEQ. ID NO:2.

18. The method of claim 14, wherein the mutated transposase protein is introduced into the host cell in step (b) by electroporation.

19. The method of claim 18, wherein the transposon that contains the transgene is introduced into the host cell in step (b) before the mutated transposase protein.

20. The method of claim 14, wherein the mutated transposase is soluble at a concentration of 20 mg/mL.

21. The method of claim 14, wherein the mutant transposase protein is degraded within 48 hours from when it is introduced into the host cell, thereby rapidly clearing transposase activity from the host cell.

22. The method of claim 14, wherein insertion efficiency of the transgene into the genome is dose dependent on the amount of the variant transposase introduced into the cell, whereby the number of insertions per genome and the number of positive clones obtained can be controlled.

23. The method of claim 14, wherein the transgene encodes a chimeric antigen receptor (CAR).

24. The method of claim 14, wherein a product obtained according to the method is configured for administration into a human subject in need thereof.
